(19) United States
(12) Patent Application Publication
(10) Pub. No.: US 2024/0315550 A1
(43) Pub. Date: Sep. 26, 2024

(54) HEAD-MOUNTED AUTOMATED OPTOMETRIC SYSTEM WITH DIGITAL VISUAL CORRECTION

(71) Applicant: iCRx, Inc., Tucson, AZ (US)

(72) Inventors: Adoum Mahamat, Tucson, AZ (US); Erol Ozgur, Tucson, AZ (US)

(21) Appl. No.: 18/189,069

(22) Filed: Mar. 23, 2023

(51) Int. Cl. A61B 3/10; A61B 3/00; A61B 3/18

(52) U.S. Cl. CPC A61B 3/1015; A61B 3/0008; A61B 3/0025; A61B 3/0083; A61B 3/18; A61B 2505/07

(57) ABSTRACT

A head-mounted optometric system for correction of an eye of a patient. The system may comprise a light source configured to generate light, a mirror configured to reflect light generated by the light source towards a beam splitter that is configured to split light into a first beam directed into the eye of the patient and a second beam directed into a wavefront sensor. The system may further comprise the wavefront sensor configured to compare light reflected from the eye of the patient and the second beam to measure errors, and a digital correction system coupled to the wavefront sensor, configured to generate and apply a processed image for correcting the eye of the patient.

### BACKGROUND OF THE INVENTION

Eye care is an essential medical field. The gold standard in eye care is a device called a phoropter, which includes a large set of optical lenses that are flipped in front of the patient's eyes by the operator, to determine the best visual acuity.

An automated phoropter, or autophoropter, is a device that is capable of objectively measuring the refractive errors, and automatically applying a correction. It is possible to use a tunable lens system (TLS) to perform the visual correction. It is also possible to miniaturize the autophotopter into a head-mounted system that has a front end for sensing and correction, and a back end in the Cloud for calculations and data management. This invention paves the way for people to obtain eye exams at the comfort of their homes. The system has a lower cost compared to a table-top autophoropter system.

The head-mounted automated phoropter system is compact and lightweight; yet, its most critical part, the TLS, is optically, mechanically, and electrically complex, and the cost of its manufacturing is significantly high. Therefore, it is desirable to find an alternative way to perform the visual correction. Spatial Light Modulators (SLMs) are also being used to generate corrected images, but they have low resolution and are also monochromatic, which may lead to chromatic aberrations in the formed image.

The emerging technology of computer-generated holograms, together with a Cloud-based Graphics Processing Unit (GPU) has an unprecedented potential to replace the TLS. This will further reduce the cost of the head-mounted optometric system and reduce its overall complexity. Higher-order aberrations other than defocus and astigmatism can be corrected on-site, which will increase the applicability and therefore the commercial potential of the device. This way of digital visual correction in a head-mounted system is a unique approach and therefore is the topic of this disclosure.

### FIELD OF THE INVENTION

The present invention relates to a home-based or point-of-care head-mounted visual acuity measurement and correction system that can deliver eye prescriptions within minutes without visiting an eye doctor's office. The invention is a modular automated measurement system that performs a digital correction in the image observed by the patient.

### BRIEF SUMMARY OF THE INVENTION

It is an objective of the present invention to provide systems and methods that allow for a home-based or point-of-care head-mounted visual acuity measurement and correction system that can deliver eye prescriptions within minutes without visiting an eye doctor's office, as specified in the independent claims. Embodiments of the invention are given in the dependent claims. Embodiments of the present invention can be freely combined with each other if they are not mutually exclusive.

The present invention features a head-mounted optometric system for digital correction of an eye of a patient. In some embodiments, the system may comprise a light source configured to generate light, a mirror configured to reflect light generated by the light source towards a beamsplitter, and the beamsplitter configured to split light into a first beam and a second beam. The first beam may be directed into the eye of the patient. The second beam may be directed into a wavefront sensor. The system may further comprise the wavefront sensor configured to compare light reflected from the eye of the patient and the second beam to measure one or more wavefront errors, and a digital correction system communicatively coupled to the wavefront sensor, configured to generate an image comprising a correction to the eye of the patient, and applying the correction to the eye of the patient.

The present invention features a method for digital correction of an eye of a patient. In some embodiments, the method may comprise actuating a light source to generate light, reflecting, by a mirror, the light generated by the light source, and splitting, by a beamsplitter, the light reflected by the mirror into a first beam and a second beam. The first beam may be directed towards an eye of the patient, and the second beam may be directed towards a wavefront sensor. The method may further comprise comparing, by the wavefront sensor, light reflected from the eye of the patient and the second beam to measure one or more wavefront errors, transmitting the one or more wavefront errors to a digital correction system, generating, by the digital correction system, an image comprising a correction to the eye of the patient, and applying the correction to the eye of the patient.

In the related invention of the automated phoropter, as shown in FIG. 1, the light is emitted from a light source, preferentially an LED and it is directed through the eye using a dichroic mirror and a polarizing beamsplitter. The reflected light is detected using a Shack-Hartmann Wavefront sensor (SHS), and the refractive error is compensated using the tunable lens system. Therefore, the patient observes a corrected microdisplay-generated image.

FIG. 2 demonstrates the innovation in the current disclosure by comparing it with a tabletop autophoropter and a head-mounted autophoropter. Briefly, in the tabletop autophoropter, the sensing, correction, calculation, and data processing are performed inside the instrument. In the head-mounted phoropter, the computation and data processing have been moved into the Cloud. The image correction is performed using a tunable lens system.

In the current disclosure, the head-mounted optometric system is no longer a phoropter, because it does not contain tunable lenses. It instead performs a digital correction.

The embodiment in FIG. 3 is one of the possible embodiments that perform the digital correction. The interrogation is performed the same way as the auto phoropter. However, after the wavefront errors are measured using the SHS, an image that contains the necessary correction is generated digitally, using the disclosed digital correction system.

One of the unique and inventive technical features of the present invention is the generation of a vision-correcting image in a cloud server communicatively coupled to an optometric system. Without wishing to limit the invention to any theory or mechanism, it is believed that the technical feature of the present invention advantageously provides for efficient correction of an eye of a patient while minimizing the processing power required from the physical optometric system. None of the presently known prior references or work has the unique inventive technical feature of the present invention. Furthermore, the inventive technical features of the present invention contributed to a surprising result. For example, one skilled in the art would implement a tunable lens system to allow for the measurement of a patient's visual acuity in order to diagnose and correct issues in a patient's eye. Surprisingly, the present invention is able to obviate the tunable lens system while efficiently and accurately measuring and correcting a patient's vision through processing power of the cloud server.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 is a schematic of a prior art head-mounted system, which includes TLS. The invention disclosed here has been formed by replacing the microdisplay and the TLS with a digital correction system.

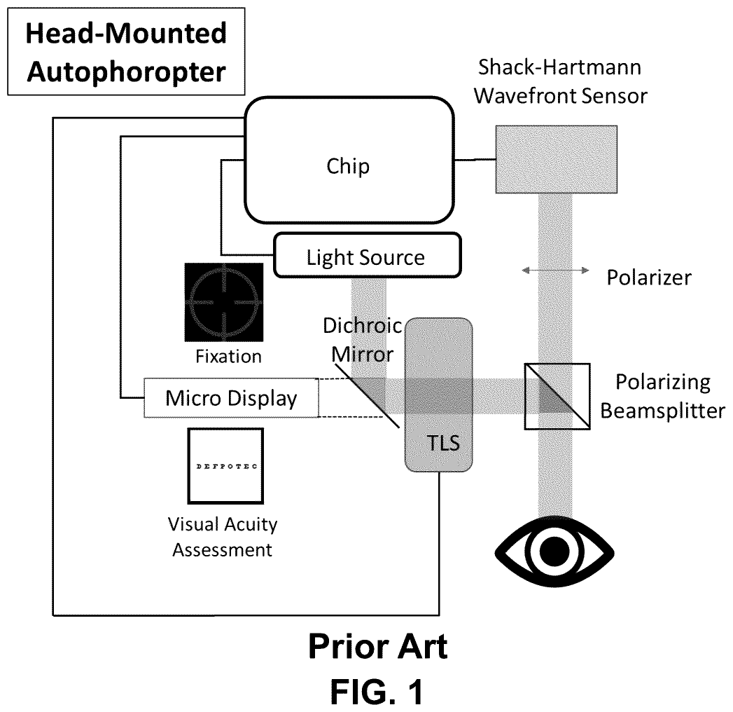

FIG. 2 shows a comparison of prior art head-mounted systems in comparison to the present invention.

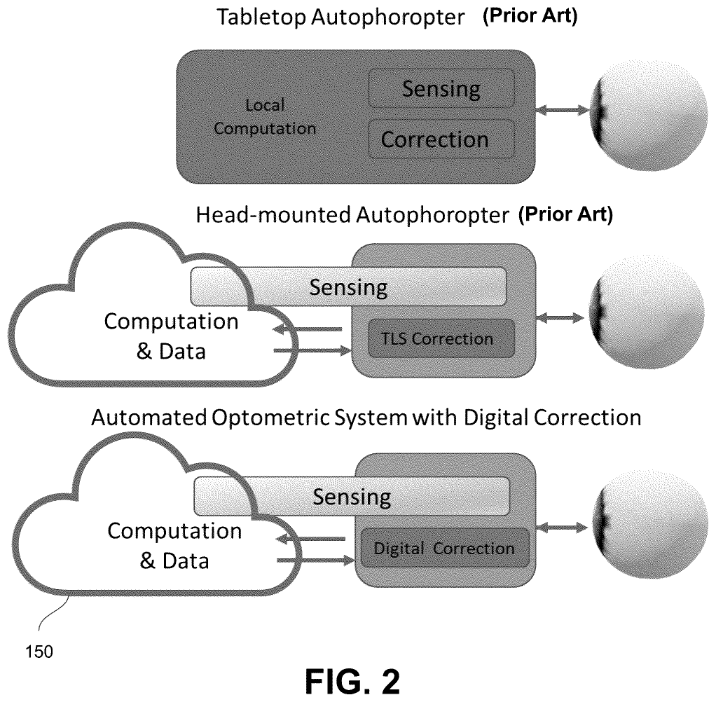

FIG. 3A is one possible embodiment of the head-mounted automated optometric system with digital visual correction.

FIG. 3B shows a flow chart of a method for digital correction of an eye of a patient, as disclosed in the present application.

### DETAILED DESCRIPTION OF THE INVENTION

Following is a list of elements corresponding to a particular element referred to herein:
100 system
110 light source
120 mirror
130 beamsplitter
140 wavefront sensor
150 digital correction system
160 communication chip

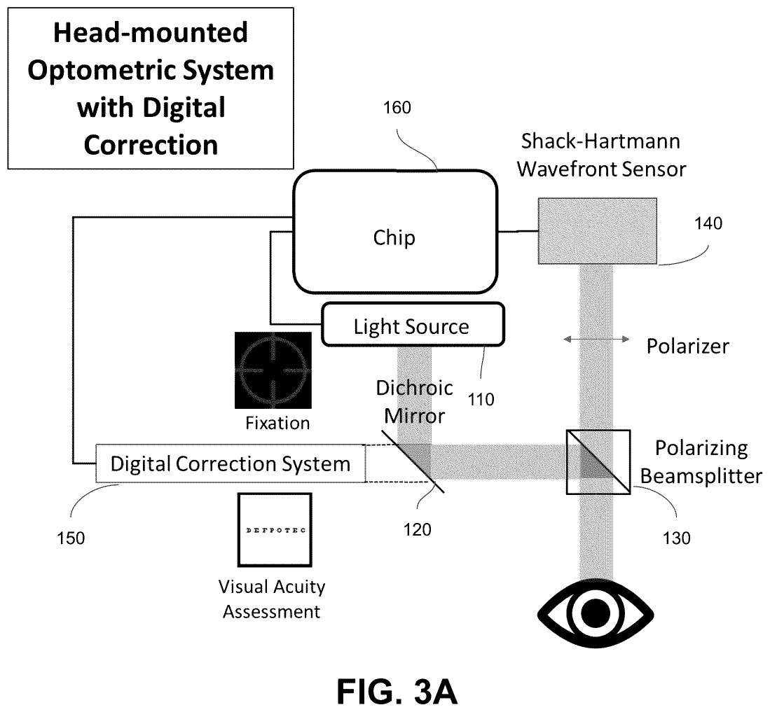

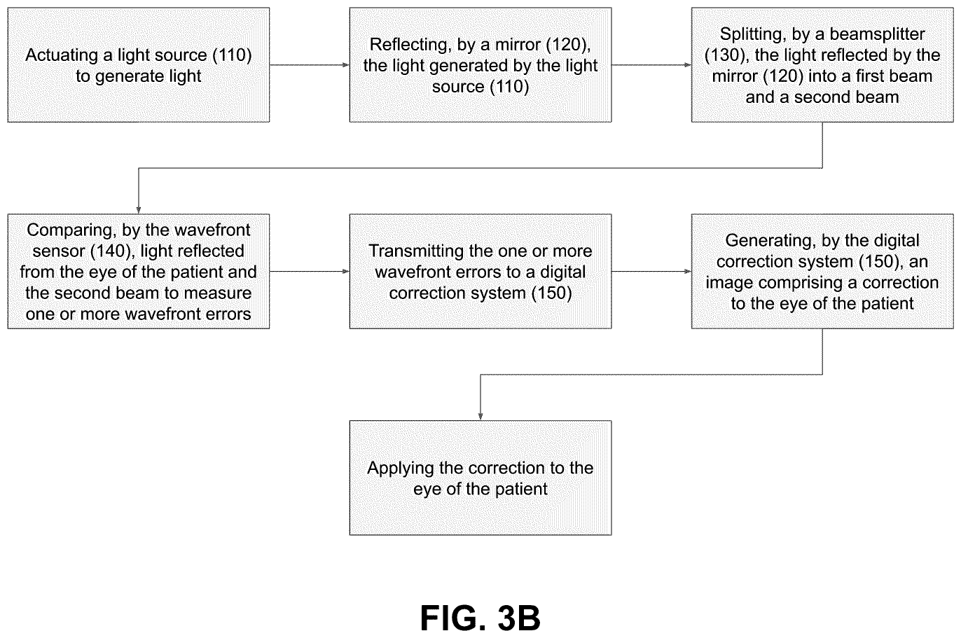

Referring now to FIG. 3A, the present invention features a head-mounted optometric system (100) for digital correction of an eye of a patient. In some embodiments, the system (100) may comprise a light source (110) configured to generate light, a mirror (120) configured to reflect light generated by the light source (110) towards a beamsplitter (130), and the beamsplitter (130) configured to split light into a first beam and a second beam. The first beam may be directed into the eye of the patient. The second beam may be directed into a wavefront sensor (140). The system (100) may further comprise the wavefront sensor (140) configured to compare light reflected from the eye of the patient and the second beam to measure one or more wavefront errors, and a digital correction system (150) communicatively coupled to the wavefront sensor (140), configured to generate a preprocessed image comprising a correction to the eye of the patient, and applying the correction to the eye of the patient.

In some embodiments, the wavefront sensor (140) may comprise a Shack-Hartmann sensor. In some embodiments, the mirror (120) may comprise a dichroic mirror. In some embodiments, the beamsplitter (130) may comprise a polarizing beamsplitter. In some embodiments, the system (100) may further comprise a polarizer disposed between the beamsplitter (130) and the wavefront sensor (140). The polarizer may be configured to polarize the second beam and the light reflected from the eye of the patient. The digital correction system (150) may comprise a cloud server comprising a processor capable of executing computer-readable instructions, and a memory component comprising computer-readable instructions for generating the image comprising the correction to the eye of the patient, and applying the correction to the eye of the patient. In some embodiments, the system (100) may further comprise a communication chip (160) communicatively coupled to the cloud server. The communication chip (160) may be configured to transmit the one or more wavefront errors and initial corrections to the cloud server In some embodiments, the wavefront sensor (140) may be configured to measure fixation and visual acuity of the eye of the patient.

Referring now to FIG. 3B, the present invention features a method for digital correction of an eye of a patient. In some embodiments, the method may comprise actuating a light source (110) to generate light, reflecting, by a mirror (120), the light generated by the light source (110), and splitting, by a beamsplitter (130), the light reflected by the mirror (120) into a first beam and a second beam. The first beam may be directed towards an eye of the patient, and the second beam may be directed towards a wavefront sensor (140). The method may further comprise comparing, by the wavefront sensor (140), light reflected from the eye of the patient and the second beam to measure one or more wavefront errors, transmitting the one or more wavefront errors to a digital correction system (150), generating, by the digital correction system (150), a preprocessed image comprising a correction to the eye of the patient, and applying the correction to the eye of the patient.

In some embodiments, the wavefront sensor (140) may comprise a Shack-Hartmann sensor. In some embodiments, the mirror (120) may comprise a dichroic mirror. In some embodiments, the beamsplitter (130) may comprise a polarizing beamsplitter. In some embodiments, The method may further comprise a polarizer disposed between the beamsplitter (130) and the wavefront sensor (140) polarizing the second beam and the light reflected from the eye of the patient. In some embodiments, the digital correction system (150) comprises a cloud server comprising a processor capable of executing computer-readable instructions, and a memory component comprising computer-readable instructions for generating the image comprising the correction to the eye of the patient, and applying the correction to the eye of the patient. In some embodiments, a communication chip (160) communicatively coupled to the cloud server transmits the one or more wavefront errors and initial corrections to the cloud server. In some embodiments, the correction may be generated at least partially in the local head-mounted portion of the system, and the at least partial correction may be transmitted to the cloud server to complete, verify, validate, and/or optimize the said correction. In some embodiments, the data collected by the wavefront sensor (140) may be preprocessed in the local head-mounted system before transmitting to the cloud server to reduce data size. In these embodiments, the local head-mounted system may comprise a processor capable of executing computer-readable instructions and a memory component operatively coupled to the processor, comprising computer-readable instructions for at least partially generating the correction and/or pre-processing the raw data from the wavefront sensor (140). The wavefront sensor (140) may be configured to measure fixation and visual acuity of the eye of the patient.

In some embodiments, the image created by the digital correction system will be an image that will be perceived as a corrected image with the existing optical aberration of the eye. The system projects collimated light into the visual system. Upon reflecting off the eye, the wavefront will pick up some aberrations due to the visual system's refractive errors. The system will digitally examine and correct the wavefront through the use software. The entire correction is done through software only.

The computer system can include a desktop computer, a workstation computer, a laptop computer, a netbook computer, a tablet, a handheld computer (including a smartphone), a server, a supercomputer, a wearable computer (including a SmartWatch™), or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R.F, Bluetooth, storage media, computer buses, etc., or any suitable combination of the foregoing. Computer program code for carrying out operations for aspects of the present disclosure may be written in any combination of one or more programming languages, including an object oriented programming language such as Java, Smalltalk, C #, Ruby, or the like, conventional procedural programming languages, such as Matlab, Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft®.NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation®; the Mac OS X operating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A head-mounted optometric system (100) for digital correction of an eye of a patient, the system (100) comprising:
a. a light source (110) configured to generate light;
b. a mirror (120) configured to reflect light generated by the light source (110) towards a beamsplitter (130);
c. the beamsplitter (130) configured to split the light into a first beam and a second beam, wherein the first beam is directed into the eye of the patient, wherein the second beam is directed into a wavefront sensor (140);
d. the wavefront sensor (140) configured to compare light reflected from the eye of the patient and the second beam to measure one or more wavefront errors; and
e. a digital correction system (150) communicatively coupled to the wavefront sensor (140), comprising a cloud server comprising a processor capable of executing computer-readable instructions, and a memory component comprising computer-readable instructions for generating the image comprising the correction to the eye of the patient, and applying the correction to the eye of the patient.

2. The system (100) of claim 1, wherein the wavefront sensor (140) comprises a Shack-Hartmann sensor.

3. The system (100) of claim 1, wherein the mirror (120) comprises a dichroic mirror.

4. The system (100) of claim 1, wherein the beamsplitter (130) comprises a polarizing beamsplitter.

5. The system (100) of claim 1 further comprising a polarizer disposed between the beamsplitter (130) and the wavefront sensor (140), wherein the polarizer is configured to polarize the second beam and the light reflected from the eye of the patient.

6. The system (100) of claim 1 further comprising a communication chip (160) communicatively coupled to the wavefront sensor (140) and the cloud server, wherein the communication chip (160) is configured to transmit the one or more wavefront errors to the cloud server.

7. The system (100) of claim 1, wherein the wavefront sensor (140) is configured to measure fixation and visual acuity of the eye of the patient.

8. A method for digital correction of an eye of a patient, the method comprising:
a. actuating a light source (110) to generate light;
b. reflecting, by a mirror (120), the light generated by the light source (110);
c. splitting, by a beamsplitter (130), the light reflected by the mirror (120) into a first beam and a second beam, wherein the first beam is directed towards an eye of the patient, wherein the second beam is directed towards a wavefront sensor (140);
d. comparing, by the wavefront sensor (140), light reflected from the eye of the patient and the second beam to measure one or more wavefront errors;
e. transmitting the one or more wavefront errors to a digital correction system (150) comprising a cloud server;
f. generating, by the digital correction system (150), a processed image comprising a correction to the eye of the patient; and
g. applying the correction to the eye of the patient.

9. The method of claim 8, wherein the wavefront sensor (140) comprises a Shack-Hartmann sensor.

10. The method of claim 8, wherein the mirror (120) comprises a dichroic mirror.

11. The method of claim 8, wherein the beamsplitter (130) comprises a polarizing beamsplitter.

12. The method of claim 8 further comprising polarizing, by a polarizer disposed between the beamsplitter (130) and the wavefront sensor (140), the second beam and the light reflected from the eye of the patient.

13. The method of claim 8, wherein the cloud server comprises a processor capable of executing computer-readable instructions, and a memory component comprising computer-readable instructions for generating the image comprising the correction to the eye of the patient, and applying the correction to the eye of the patient.

14. The method of claim 13, wherein a communication chip (160) communicatively coupled to the wavefront sensor (140) and the cloud server transmits the one or more wavefront errors to the cloud server.

15. The method of claim 8, wherein the wavefront sensor (140) is configured to measure fixation and visual acuity of the eye of the patient.

16. A head-mounted optometric system (100) for digital correction of an eye of a patient, the system (100) comprising:
a. a light source (110) configured to generate light;
b. a dichroic mirror (120) configured to reflect light generated by the light source (110) towards a polarizing beamsplitter (130);
c. the polarizing beamsplitter (130) configured to split light into a first beam and a second beam, wherein the first beam is directed into the eye of the patient, wherein the second beam is directed into a Shack-Hartmann sensor (140);
d. the Shack-Hartmann sensor (140) configured to compare light reflected from the eye of the patient and the second beam to measure one or more wavefront errors;
e. a polarizer disposed between the polarizing beamsplitter (130) and the Shack-Hartmann sensor (140) such that second beam and the light reflected from the eye of the patient are polarized by the polarizer;
f. a communication chip (160) communicatively coupled to the Shack-Hartmann sensor (140) and a cloud server, configured to transmit the one or more wavefront errors to the cloud server; and
g. the cloud server communicatively coupled to the Shack-Hartmann sensor (140) by the communication chip (160), comprising a processor capable of executing computer-readable instructions, and a memory component comprising computer-readable instructions for:
i. generating an image comprising a correction to the eye of the patient, and
ii. applying the correction to the eye of the patient.

17. The system (100) of claim 16, wherein the Shack-Hartmann sensor (140) is configured to measure fixation and visual acuity of the eye of the patient.
